(19) United States
(12) Plant Patent Application Publication
(10) Pub. No.: US 2012/0240299 P1
(43) Pub. Date: Sep. 20, 2012

(54) Interspecific cornus hybrid designated KV10-105v1

(50) ;

(75) Inventor: Dennie Hill, Huntland, TN (US)

(73) Assignee: Rutgers, The State University of New Jersey, New Brunswick, NJ (US)

(21) Appl. No.: 13/385,877

(22) Filed: Mar. 12, 2012

Related U.S. Application Data
(60) Provisional application No. 61/465,140, filed on Mar. 15, 2011.

(51) Int. Cl. A01H 5/00

(52) U.S. Cl. PLT/220

(57) ABSTRACT

An interspecific hybrid of Cornus kousa and Cornus florida, comprising a bud mutation of the tree covered by PP 07207. The mutation is a vigorous dogwood tree, exhibiting variegated foliage comprising green leaves with a white margin and dispersed green and mottled hues of green and white in several shades during the growing season and brilliant fall foliage with multiple shades of brilliant red-purple margins with dispersed shades of violet blue.

### CROSS REFERENCE TO RELATED APPLICATIONS

This application claims the benefit of priority of provisional patent application Ser. No. 61/465,104, filed on Mar. 15, 2011, confirmation number 5018, and entitled “Interspecific Cornus hybrid designated KV10-105v1,” which is incorporated herein in its entirety by reference.

### BACKGROUND OF THE INVENTION

This invention relates to dogwood trees and particularly to the selection of a mutation of a F₁ hybrid, after the interspecific hybridization of an unnamed plant of Cornus kousa× a plant of Cornus florida ‘Sweetwater’.

The particular seedlings hereof were selected from certain progeny grown in a cultivated area in Huntland, Tenn. and, as a result, have in turn been asexually reproduced by grafting. The reproduction and actual growth and selection of the new cultivar took place in the vicinity of Huntland, Tenn. and has been found to be distinctive as to its variegated foliage.

Plants of this hybrid are vigorous and upright in habit, and are believed to be floriferous. The broad, rounded, overlapping bracts are believed to provide a floral display from about mid May to early June in Zones 6-7. The plants are believed to be free of infestation by the dogwood borer and are not expected to exhibit “dogwood decline”.

The plant is capable of being grown wherever plants of C. kousa and C. florida are grown although they have not been tested in any widespread areas by me or under my direction.

This mutation has been asexually reproduced by bud grafting so as to determine that it does in fact come true in successive generations. In my estimation, it has desirable commercial attractiveness due to the variegation of the leaves both during the growing season and in the fall.

In the following detailed description which includes some comments in respect to various aspects of the plant, there is provided in addition, drawings which disclose the foliage in color and the colors are referred to the RHS Colour Chart published by The Royal Horticulture Society (1995) for individual notations and comments.

### BRIEF DESCRIPTION OF THE DRAWINGS

FIG. 1 discloses a tree of my new cultivar in the second full year of growth;

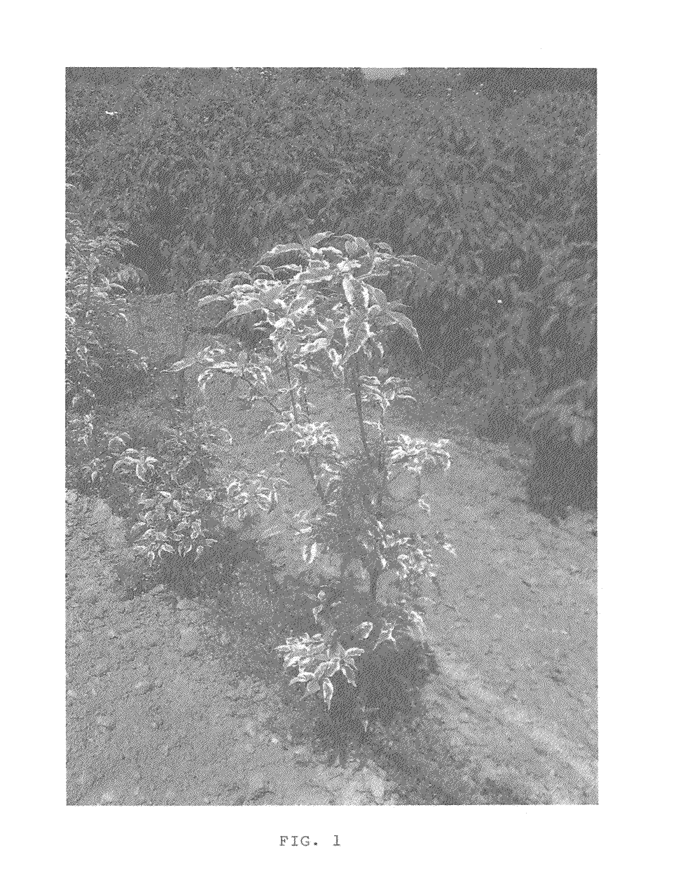

FIG. 2 discloses the variegated leaves of this new cultivar during the growing season;

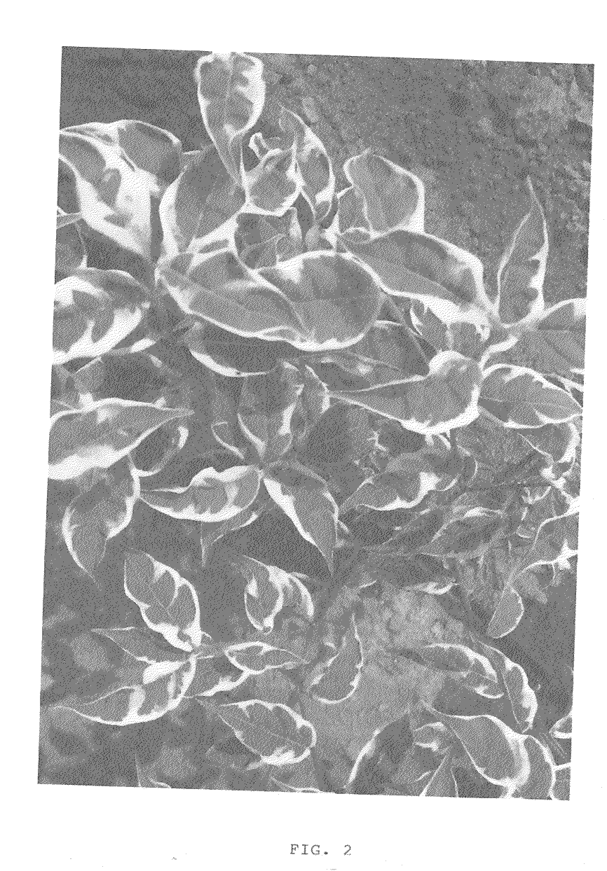

FIG. 3 shows the variegated coloration of the leaves in autumn;

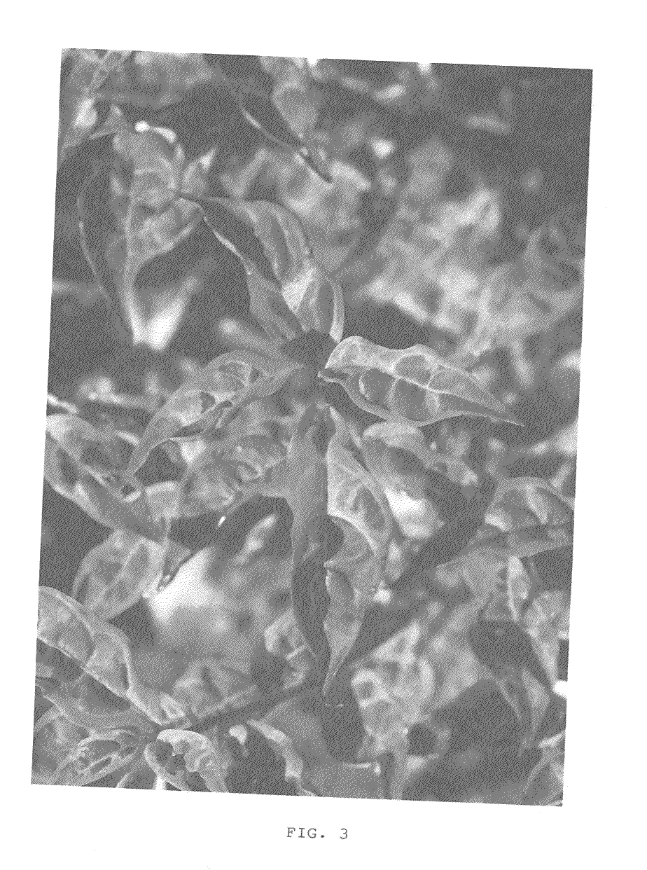

FIG. 4 shows the variegated leaves in transition from the growing season to the autumn; and

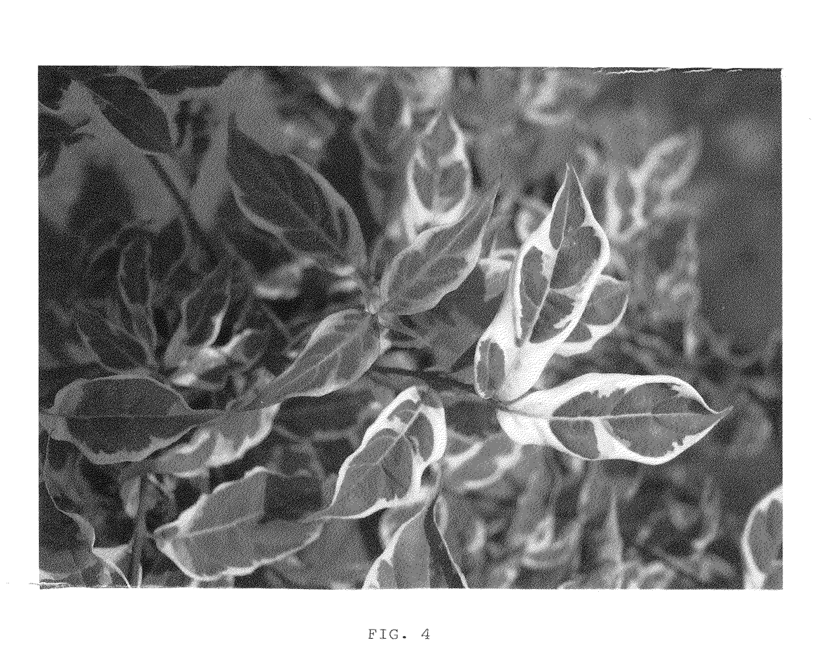

FIG. 5 shows the commencement of budding of a floral bract.

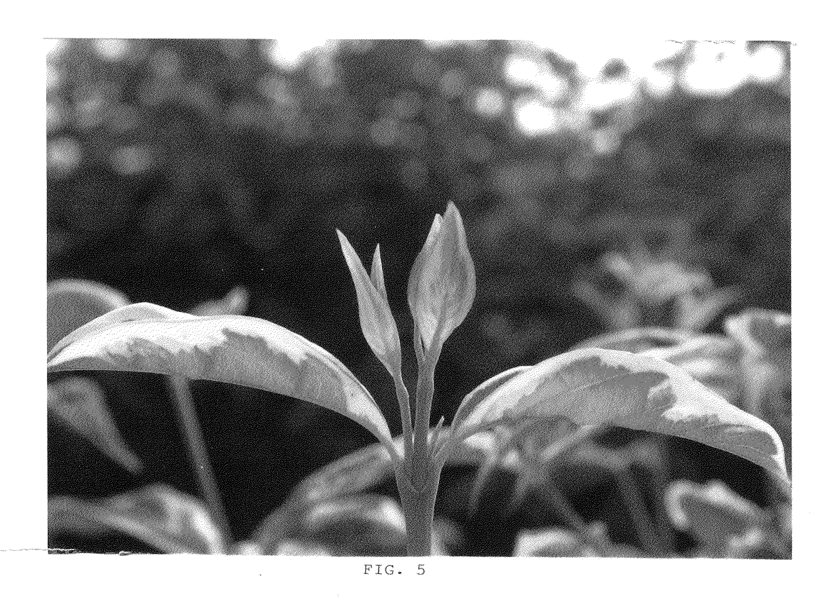

The coloration is shown in the drawings made from digital photographs which thus reproduce the same as near as is possible to the colors of the actual plant with the notations referred thereto. Colors of foliage and other plant parts may vary from year to year depending on, among other factors, horticultural practices, light conditions, air temperature, soil fertility, etc.
Botanical description and variety denomination:
   Classification.—Botanic name: F₁ interspecific hybrid of C. kousa×C. florida.
   Commercial name.—KV10-105v1.
Parentage:
   Seedling.—F₁ interspecific hybrid.
   Seed parent.—Unnamed seedling of Cornus kousa ‘Hance’.
   Pollen parent.—Cornus Florida L. cv. ‘Sweetwater’.
Plant:
   Method of asexual propagation.—Grafting buds from a variegated branch mutant onto Cornus kousa seedling understock, holds its distinguishing leaf variegation characteristics through asexual propagation by means as grafting, budding, or rooting of softwood or semi-hardwood stem cuttings.
   Tree.—Vigorous — upright and spreading, or full — dense — somewhat vase-shaped when young, but broad and full as an older plant — hardy — very productive, i.e. believed to be very floriferous (but highly sterile).
   Trunk.—Medium — smooth when young, but believed to become somewhat shaggy with age.
   Branches.—Medium — smooth.
   Lenticels.—Numerous — medium.
Leaves: Abundant.
   Length.—10-14 cm (average about 12.3).
   Width.—4.8-6.6 cm (average about 5.4) at widest point. Medium size.
   Color.—Color during growing season: white margins with varying shades between RHS 157C and RHS 155A; interior dispersed green and mottled hues of green in several shades of RHS 135D, 138C, 138D, 139C and 139D abaxial and adaxial. Color of leaves as they commence transition to fall coloration: margins, abaxial and adaxial, between white RHS 155A and light shades of red-purple 62C, and dispersed shades of red-purple 67A throughout. Color in late summer and fall: abaxial-brilliant red-purple margins with shades of RHS 62A through RHS 62D, and dispersed shades of violet blue RHS 91A through RHS 91D; adaxial- 138B-138D.
   Leaf shape.—Elliptic, with acuminate tip and cuneate base.
   Margin.—Crenate.
   Petiole.—6-13 mm in length (average 10.3 mm).
   Texture.—Velvety, corrugated. The remaining observable horticultural traits of KV10-105v1 are believed to be similar to those of the dogwood described in expired plant patent PP7207. However, not all of these traits have yet been verified.
Flower buds: The true flowers are believed to be small and numerous, borne in flower heads. Typically, the true flowers will be relatively inconspicuous. The floral display will be provided by the large floral bracts that immediately subtend the flower heads.
   Hardy.—U.S.D.A. Plant Hardiness Zone 6a (−10 degrees F.).
   Color.—Margins RHS 41B; Interior dispersed shades of greyed-green RHS 197A −197C.
Bracts:
   Color.—(anticipated) Upper surface: R.H.S. 49A (Red Group). Lower surface: R.H.S. 49D (Red Group).
   Size.—Likely to be larger in size than those of the seed parent and equal in size to those of the pollen parent. When fully expanded the diameter of the inflorescence from tip to tip of the opposing inner bracts should be about 116 mm; the diameter of the inflorescence as measured from tip to tip of the opposing outer bracts would be about 105 mm. The average length of the inner bracts — approximately 56 mm. The average length of the outer bracts — approximately 50 mm. The average width of the inner bracts at their widest point — probably about 40 mm. The average width of the outer bracts at their widest point — about 46 mm. Bract length and width will vary from year to year, but the inner bracts will most likely be longer and narrower than the outer bracts each year.
   Shape.—Bracts are expected to be sessile, nearly rounded to obovate with short, acute tips and tapered bases. The inner bracts are expected to be longer and narrower than the outer, or lower bracts. The margins of the basal one-third of adjacent bracts typically would overlap.
   Peduncle length.—Each flower head may be borne on a peduncle, the average length expected to be about 5 cm at the time of flowering, whereas those of the C. kousa parent average approximately 7 cm in length and those of the C. florida parent average about 3 cm at the time of flowering. Peduncle length will vary from year to year but the relative difference between the three plants (two parents and the variegated hybrid) should be quite consistent.
   Flowers.—date of first and full bloom: Anthesis of the tiny, relatively inconspicuous true flowers generally commences two to four days following the onset of the ornamental display of the large floral bracts. The average number of true flowers per flower head is expected to be intermediate to that for the C. kousa parent (average 49) and that for the C. florida parent (average 29). Flowering period will be early compared with plants of C. kousa and late compared with plants of C. florida. Period of floral display (floral bracts) is anticipated to be intermediate to that of plants of the parent species; i.e., occurring in mid-May, commencing one to three days after completion of the floral display (floral bracts) of most plants of C. florida and ending a few days prior to the onset of the floral display (floral bracts) of most plants of C. kousa.
Reproductive organs: The anticipated properties of the reproductive organs are likely to be:
Stamens:
   Number per flower.—4.
   Filament size.—4-5 mm long and 0.25-0.30 mm wide.
   Anther size.—approximately 1 mm long.
   Pistil.—Number — 1.
   Style length.—approximately 1.5 mm long and 0.5 mm diameter.
   Stigma height.—approximately 0.25 mm.
Fruit: Fully developed fruit have not been observed, as plants of this new F₁ interspecific hybrid are highly sterile. However, some flower heads may persist throughout the growing season and, occasionally, the fleshy portion of small, parthenocarpic (seedless) fruit will develop, albeit incompletely. However, there will be no formation of a syncarp as in C. kousa, as the flowers in this new hybrid are distinct and separate in the flower head, or on the receptacle.
Resistance to: Insects. — such as dogwood borer-believed to be good. Diseases. — believed to be equivalent to that of the dogwoods covered by PP7207.

I claim:

1. A new and distinct cultivar of dogwood tree, substantially as herein shown and described, characterized particularly as to novelty by exhibiting variegated green leaves with a white margin and dispersed green and mottled hues of green and white in several shades during the growing season and brilliant fall foliage with multiple shades of brilliant red-purple margins with dispersed shades of violet blue.
